(19) United States
(12) Patent Application Publication
(10) Pub. No.: US 2010/0305071 A1
(43) Pub. Date: Dec. 2, 2010

(54) METHODS FOR IMPROVING HEALING OF AN ORAL LESION USING A GLYCEROPHOSPHATE SALT

(75) Inventor: Alan E. Kligerman, Egg Harbor Township, NJ (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: PRELIEF INC., Egg Harbor Township, NJ (US)

(21) Appl. No.: 12/674,773

(22) PCT Filed: Aug. 28, 2008
(86) PCT No.: PCT/US08/74551

§ 371 Date: May 17, 2010

Related U.S. Application Data
(60) Provisional application No. 60/968,928, filed on Aug. 30, 2007.

(51) Int. Cl. A61K 31/661; A61P 1/02; A61P 9/00; A61P 9/10; A61P 11/00

(52) U.S. Cl. 514/129

(57) ABSTRACT

Glycerophosphate salts have been found to hasten the healing and rapid scaling of lesions of the mouth. Methods are provided for improving healing of an oral lesion or preventing diseases or conditions related to an oral lesion using a glycerophosphate salt. In particular, methods are provided for preventing diseases or conditions related to an oral lesion using calcium glycerophosphate (CGP).

### BACKGROUND OF THE INVENTION

Every year, large numbers of patients undergo various dental procedures, ranging from simple tooth cleaning to complex dental surgery. Many of these procedures cause a breach in the oral/gingival mucosa, causing pain and providing opportunity for infection by one or more of the many species of microbes that colonize the mouth.

Therefore, there is a need to develop a novel, simple, and relatively inexpensive means to improve healing of oral lesions, and thus lessen the duration of pain and prevent diseases or conditions related to oral lesions.

### BRIEF SUMMARY OF THE INVENTION

It is now discovered that a glycerophosphate salt can be used to improve the healing of oral lesions. This discovery provides a novel means to prevent diseases or conditions related to an oral lesion in a subject.

In one general aspect, an embodiment of the present invention relates to a method of improving healing of an oral lesion in a subject. The method comprises: (a) locating the oral lesion in the mouth of the subject; and (b) applying an effective amount of a glycerophosphate salt to the oral lesion in a form of dry powder, granule or gel to form a coating over the oral lesion, or in a mouthwash containing about 0.5% to about 75% by weight of a glycerophosphate salt.

In one embodiment, the present invention relates to a method of preventing a disease or condition related to an oral lesion in a subject. The method comprises improving healing of the oral lesion in the subject using methods according to embodiments of the present invention.

In another general aspect, an embodiment of the present invention relates to a kit for improving healing of an oral lesion in a subject. The kit comprises a glycerophosphate salt and instructions for using the glycerophosphate salt to improve healing of an oral lesion in a subject.

Other aspects, features and advantages of the invention will be apparent from the following disclosure, including the detailed description of the invention and its preferred embodiments and the appended claims.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must be noted that as used herein and in the appended claims, the singular forms “a,” “an,” and “the” include plural reference unless the context clearly dictates otherwise.

The newly discovered function of glycerophosphate salt to hasten or improve the healing and sealing of oral lesions provides a novel means to prevent diseases or conditions related to oral lesions, such as those resulting from dental procedures. This is of particular importance to patients whose overall medical condition (e.g. those with diabetes, HIV, or taking various immunosuppressive therapeutic regimens) delays normal wound healing. Embodiments of the present invention relate to a method of accelerating or improving healing of an oral lesion, thus reducing the likelihood and preventing a disease or condition related to the oral lesion in a subject. The method comprises locating an oral lesion in the mouth of the subject and applying an effective amount of a glycerophosphate salt to the lesion to accelerate the healing of the lesion, thereby restoring the original integrity of the normal natural “dam”, and thereby preventing the disease or condition related to the oral lesion.

Methods according to embodiments of the present invention are effective in improving healing of an oral lesion in a subject.

As used herein an “oral lesion”, a “lesion in the mouth,” “lesion of the mouth,” and “mouth lesion,” are used interchangeably and all refer to any wound, injury, pathological change or condition, or traumatic discontinuity of tissue of the mouth. The lesion can be located anywhere within or associated with the mouth, such as the gum, insides of cheeks, throat, palate, tongue, or lips of the mouth. Examples of lesions in the mouth can be a sore, an ulcer, an oral abscess, an oral candidiasis, or any oral wound. Mouth lesions can result from irritation, routine daily activities such as chewing food, tooth brushing, or dental flossing, any dental or oral procedure such as tooth extraction, implant, restoration, etc., any oral surgery, or any other diseases or conditions.

As used herein, “improving healing of an oral lesion” refers to faster, better, or both faster and better, healing of the oral lesion.

Methods according to embodiments of the present invention are effective in preventing any one or more of the disease or condition related to an oral lesion describe above.

As used herein, the term a “disease or condition related to an oral lesion” includes any disease, disorder or condition that has been caused, aggravated, or otherwise associated with an oral lesion. It needs to be emphasized that a disease or condition related to an oral lesion may or may not be caused, aggravated, related to, or otherwise associated with a mouth disease, such as an oral infection. Normal, unremarkable oral micro-organisms exist in the mouth that in and of themselves engender no oral or other symptoms, but which when allowed to breach the periodontal barrier, e.g., via an oral lesion, to circulate in the blood systemically, have the inherent quality of creating diseases, disorders, or conditions elsewhere in the body. A disease or condition related to an oral lesion includes, but is not limited to, oral infection per se. Examples of diseases or conditions related to an oral lesion include, but are not limited to, a mouth disease or condition, a cardiovascular disease or condition, a poorly controlled diabetes, or preterm birth. The mouth disease or condition includes, but is not limited to, a gum disease such as gingivitis, periodontitis or acute necrotizing ulcerative gingivitis; or a sore located anywhere within or associated with the mouth, such as on the insides of cheeks, throat, palate, tongue, or lips. The cardiovascular disease or condition includes, but is not limited to, endocarditis, stroke, atherosclerotic plaques in the arteries, or mitral valve prolapse.

As used herein, the phrase “preventing a disease or a condition related to an oral lesion” means to interdict, palliate, alleviate, reduce, decrease, or prevent the disease or condition related to an oral lesion. “Preventing a disease or a condition related to an oral lesion” can be a prevention of the further development or aggravation of an existing disease or condition related to an oral lesion. “Preventing a disease or a condition related to an oral lesion” can also be a prevention before symptoms of the disease or condition related to an oral lesion are developed or observable. “Preventing a disease or a condition related to an oral lesion” results in a clinically observable beneficial effect. The clinically observable beneficial effect can be a situation that symptoms of an existing disease or condition related to an oral lesion are prevented from further development or aggravation, or develop to a lesser degree than without administration of the composition of the present invention, when a composition of the present invention is administered to a subject after the symptoms are observable. The clinically observable beneficial effect can also be a situation in which symptoms of a disease or a condition related to an oral lesion are prevented from occurring or subsequently occur to a lesser degree than without administration of the composition of the present invention, when a composition of the present invention is administered to a subject before the symptoms are observable.

As used herein, the term “subject” refers to an animal, preferably a mammal, who/which has been the object of treatment, observation or experiment. Examples of a subject can be a human, a livestock animal (beef and dairy cattle, sheep, poultry, swine, etc.), or a companion animal (dog, cat, horse, etc).

The term “effective amount” as used herein means that amount of a glycerophosphate salt that accelerates the healing of a lesion in a mouth of a subject, thereby preventing a disease or a condition related to an oral lesion. In one embodiment of the invention, an effective amount of a glycerophosphate salt accelerates the healing of a lesion in a mouth of a subject, such that it takes less than about 10%, 20%, 30%, 40%, 50%, 60%, 70%, 80%, or 90%, preferably less than about 30-50%, of the time that would have taken to heal the lesion had the subject not received an effective amount of the glycerophosphate salt. The administration of an “effective amount” of a glycerophosphate salt to a lesion in a mouth of a subject results in a clinically observable beneficial effect as described above.

One skilled in the art will recognize that the “effective amount” of a glycerophosphate salt to be used in the instant invention can vary with factors, such as the particular subject, e.g., age, diet, health, etc., size of the oral lesion, severity and complications of the disease or condition sought to be prevented, the mode of administration of a glycerophosphate salt, the particular glycerophosphate salt used, etc. Standard procedures can be performed to evaluate the effect of the administration of a glycerophosphate salt to a subject, thus allowing a skilled artisan to determine the effective amount of the glycerophosphate salt to be administered to the subject in view of the present disclosure.

As used herein, the term “glycerophosphate salt” refers to a chemical compound that is derived from glycerophosphate, in which one or more of the hydrogens of the phosphate group of glycerophosphate are replaced by a basic radical, in particular embodiments by a metal ion. As used herein, the term “glycerophosphate” refers to an anion of a phosphoric ester of glycerol, in which a carbon atom of glycerol bonds to an oxygen atom in the phosphate group of phosphoric acid. A glycerophosphate salt can be a chiral molecule, i.e., it can exist in two forms that are nonsuperimposable mirror images. It is intended that the present invention includes within its scope the stereochemically pure isomeric forms of a glycerophosphate salt and/or their racemates.

In particular embodiments, methods of the invention utilize one or more glycerophosphate salts selected from the group consisting of calcium glycerophosphate (CGP), magnesium glycerophosphate, zinc glycerophosphate, manganese glycerophosphate, lithium glycerophosphate, cupric glycerophosphate, ferric glycerophosphate, quinine glycerophosphate, glycerophosphate disodium, glycerophosphate dipotassium, glycerophosphate barium, and glycerophosphate strontium.

The effective amount of an glycerophosphate salt takes into account of the efficacy of the particular glycerophosphate salt used. For example, extreme caution is taken to use lithium glycerophosphate in a method according to an embodiment of the present invention, because its effects on mood and its possible adverse effects on cardiac and renal function. The effective amount also takes into account of other properties of the glycerophosphate salt, such as toxicity (e.g., cupric glycerophosphate) and taste (e.g., ferric glycerophosphate and quinine glycerophosphate).

In a preferred embodiment, methods of the invention utilize calcium glycerophosphate. As used herein, the term “calcium glycerophosphate” or “CGP,” also known as “glycerophosphate calcium,” refers to a chemical compound having a molecular formula of C₃H₇CaO₆P in its anhydrous form. “CGP” can also exist as a hydrate, including the monohydrate and the dihydrate. Examples of calcium glycerophosphate include, but are not limited to, any one, or any combination of two or more of the three isomers of CGP, namely β-glycerophosphoric acid calcium salt ((HOCH₂)₂CHOPO₃Ca) and D(+) and L(−)-α-glycerophosphoric acid calcium salt (HOCH₂CH(OH)CH₂OPO₃Ca).

Calcium glycerophosphate available from various commercial sources can be used in the present invention. In one embodiment, Prelief®, a dietary supplement for use in reducing the impact of acid in foods and beverages that is available from AkPharma Inc. (Pleasantville, N.J. 08232), in either tablet or powder form, can be used in the present invention for orally administering the calcium glycerophosphate to the subject. Other commercially available CGP also includes CGP available from Astha Laboratories Pvt, Ltd, B-4, Industrial Estate, Sanathnagar, Hyderabad-18, India, and Seppic Inc., 30 Two Bridges Road, Fairfield, N.J.

In another embodiment, methods of the invention utilize a glycerophosphate salt other than calcium glycerophosphate. Calcium glycerophosphate may be contraindicated for persons with poor renal function or who are in renal failure. The presence of calcium ion may also suppress activities of certain drugs, e.g., certain antibiotic drugs, that are co-administered with the glycerophosphate salt. Therefore, methods of the invention also utilize one or more glycerophosphate salts selected from the group consisting a Na, K, Mg, or Sr salt of glycerophosphate, or any other non-calcium glycerophosphate salts described herein.

A glycerophosphate salt can be administered to a lesion of the mouth by one or more routes of administration. In one embodiment, an effective amount of a glycerophosphate salt, in a form of dry powder, granules, or gel is applied directly to the lesion via direct spray, physical transfer by finger or implement or as a coating on, e.g., gauze, to reach a final local concentration of the glycerophosphate salt of about 25%-100% by weight. For example, the final local concentration of the glycerophosphate salt can be about 25% to about 35%, about 35% to about 45%, about 45% to about 55%, about 55% to about 65%, about 65% to about 75%, about 75% to about 85%, or about 85% to about 100%, by weight. However, the final local concentration of the glycerophosphate salt is not limited to the range of 25%-100% by weight. It has been adequately observed on epidermal skin that lower concentrations of CGP, such as between 1% and 2% by weight, favorably affect apparent rhino mucosal passage inflammation and would therefore have a similar favorable effect on oral mucosa.

In another embodiment, an effective amount of a glycerophosphate salt is applied to the lesion by taking into the mouth in a mouthwash, containing about 0.5% to about 75% by weight of the glycerophosphate salt. For example, the mouthwash can contain about 5% to about 15%, about 15% to about 25%, about 25% to about 35%, about 35% to about 45%, about 45% to about 55%, about 55% to about 65%, or about 65% to about 75%, by weight of the glycerophosphate salt.

After the effective amount of a glycerophosphate salt is applied to the lesion of the mouth for a sufficient period of time, the glycerophosphate salt can be either swallowed or spit out.

How often and how long a glycerophosphate salt is administered to a subject depends on the type of treatment or prevention, how the subject responds to the glycerophosphate salt, factors associated with the subject, e.g., age, diet, health, etc., the size of the oral lesion, ability to tolerate the glycerophosphate salt, and the types of glycerophosphate salt used. A glycerophosphate salt can be administered on a regimen of one to multiple times per day. Preferably, a glycerophosphate salt is administered to the subject at intervals during the day, such as after breakfast, lunch, dinner, and upon retiring.

While not wishing to be bound by theory, a glycerophosphate salt can be used to prevent a disease or a condition related to an oral lesion at least in part due to the quicker repair to and replacement of cells occasioned by the presence of the glycerophosphate salt. The faster the healing of wounds or lesions within a mouth, the less likely there is to be systemic infection related to an oral lesion. Various observations suggest that a glycerophosphate salt functions to promote epidermal cell renewal, see for example, US2004/0037766. The quick repair and replacement of epidermal cells provide, among other things, enhanced ceramide synthesis, which hastens repair of the skin's surface and provides tighter cell-to-cell adhesion, which may prevent invasion between vulnerable cell walls of irritating substances. It is believed that calcium promotes the formation of tight junctions, specific structures that join epithelial cells together to form an effective barrier. Without calcium, such tight junctions cannot be formed.

Methods according to embodiments of the present invention are particularly desirable for a population of subjects, e.g., human beings, who are specifically vulnerable to diseases or conditions related to oral lesions because their pre-existing conditions have predisposed them to a higher risk of having or developing diseases or conditions related to oral lesions.

In one embodiment, a glycerophosphate salt can be applied to a mouth lesion of a subject having diabetes or other endocrine disorders, immune disorders, sexually transmitted diseases, virus infections, inflammatory bowel disease, neutropenia, blood disorders, etc. These subjects are prone to mouth sores or other mouth lesions and/or are often slow in the healing of the mouth lesions. For example, uncontrolled diabetes impairs neutrophils, white blood cells that are a main defense against bacterial infection. People having diabetes may have problems healing quickly after oral surgery or other dental treatment because blood flow to the site can be impaired. Any type of infection may cause blood-sugar levels to rise and the need for insulin to increase. Therefore, fastening the healing of lesions of the mouth can improve diabetic control. Under these conditions, the effective amount of glycerophosphate salt can be administered in combination with an anti-diabetic agent, such as insulin, exenatide, pramlintide, sulfonylureas, meglitinides, etc.

In another example, patients with inflammatory bowel disease (Crohn's disease or ulcerative colitis) may experience ulcers, sores and overgrowths of tissue in their mouths. Some of these patients may also experience oral fungal infections, i.e., thrush, as a medication side effect. Under these conditions, the effective amount of glycerophosphate salt can be administered in combination with one or more agents used to treat the inflammatory bowel disease, such as an anti-inflammatory drug, e.g., an anti-histamine, an immunosuppression drug, e.g., a steroid medication, or a biological medication, such as infliximab.

A drug that suppresses the immune system, such as a steroid, a glucocorticoid, can delay wound healing. Persons taking immunosuppressive therapy, for example, persons undergoing treatment for an inflammatory bowel disease, an autoimmune disease, or an organ or bone marrow transplant, may require longer time for healing of oral lesions, thus have increased risk of diseases or conditions related to oral lesions. Therefore, an effective amount of glycerophosphate salt can be administered to patients under treatment of an immunosuppression drug to prevent diseases or conditions related to oral lesions.

In another embodiment, an effective amount of glycerophosphate salt is applied to a mouth lesion of a subject having a trauma, an accident of all types, such as laceration of tissue with fork, or an auto accident, a war wound, a barroom brawl jaw punch, a recurrent oral skin problem such as cracking from xerostomia, etc.

An effective amount of glycerophosphate salt can also be applied to a mouth lesion of a subject having certain preexisting heart conditions. Certain oral bacteria can enter the blood stream via lesions in the mouth and settle on normal or abnormal heart valves or tissue weakened by a preexisting heart problem or heart condition. In these cases, the bacteria can damage or even destroy heart valves or tissue, causing endocarditis and other cardiovascular diseases or conditions related to an oral lesion. The inflammation associated with periodontitis may also play a role. Investigations have found an association between an increased level of blood vessel thickening and the presence of the bacteria found in dental plaque.

Examples of preexisting heart problems or conditions that predispose a subject to higher risks of cardiovascular diseases or conditions include, but are not limited to, an artificial (prosthetic) heart valve, a history of previous endocarditis, heart valves damaged (scarred) by conditions such as rheumatic fever, congenital heart or heart valve defects or hypertrophic cardiomyopathy, surgically constructed systemic-pulmonary shunts, mitral valve prolapse with valvular or mitral regurgitation (leakage) and/or thickened leaflets, or a heart transplant that results in a heart valve abnormality.

Accelerating or fastening the healing of a mouth lesion by administering an effective amount of glycerophosphate salt to the mouth lesion would reduce the risk and thus prevent cardiovascular diseases or conditions related to an oral lesion in subjects having such preexisting heart problems or conditions.

In yet another embodiment, a glycerophosphate salt can be applied to a mouth lesion of a subject under a chemotherapy or therapeutic radiation treatment. Certain side effects may develop from chemotherapy or therapeutic radiation treatment, including, but not limited to, painful mouth and gums, dry mouth, burning, peeling or swelling tongue, and infection. These side effects impose a large problem for persons undergoing treatment for any type of cancers in the head/neck area. Solutions/suspensions of glycerophosphate at various concentrations may be swirled around the mouth or sprayed or jetted into the mouth via e.g., a rubber bulb, to more easily reach parts of the oral cavity that may not be able to be accessed via vigorous swishing, when circumstances are too painful for the latter.

In a particular embodiment of the present invention, a glycerophosphate salt can be administered to a subject to treat and prevent an oral mucositis. Mucositis is the painful inflammation and ulceration of the mucous membranes lining the digestive tract, usually as an adverse effect of chemotherapy and radiotherapy treatment for cancer. Oral and gastrointestinal (GI) mucositis can affect up to 100% of patients undergoing high-dose chemotherapy and hematopoietic stem cell transplantation (HSCT), 80% of patients with malignancies of the head and neck receiving radiotherapy, and a wide range of patients receiving chemotherapy (Rubenstein et al., Cancer. 2004 May 1; 100(9 Suppl):2026-46). About 75-85% of bone marrow transplantation recipients experience mucositis, of which oral mucositis is the most common and most debilitating, especially when melphalan is used (Rubenstein et al., above). Oral mucositis is particularly profound and prolonged among HSCT recipients who receive total-body irradiation (Rubenstein et al., above). In grade 3 oral mucositis, the patient is unable to eat solid food, and in grade 4, the patient is unable to consume liquids as well (Rubenstein et al., above). Not only is this mucositis painful, leading to need for narcotic pain relief, Nasogastric feeds (e.g., involving a feeding tube to the stomach via the nose), gastrostomy feeds or parenteral nutrition, this severe mucositis leads to breakdown of a very important barrier to infection from oral bacteria, which can be serious and life-threatening/lethal. Alimentary track mucositis increases mortality and morbidity and contributes to rising health care costs (Rubenstein et al., above).

Administering an effective amount of a glycerophosphate salt to a patient under chemotherapy and radiotherapy treatment for cancer, will improve the healing of oral lesions, which will in turn treat or prevent oral mucositis and further prevent diseases or conditions related to oral mucositis.

In another embodiment, a glycerophosphate salt can be applied to a mouth lesion of a subject undergoing a procedure during which the subject may be more susceptible to diseases or conditions related to oral lesions. Such procedures can be, for example, certain type of implant procedure; a total joint replacement procedure; a dialysis therapy; a dental procedure; an oral surgery such as tonsillectomy or adenoidectomy; an incision and drainage of infected oral tissue; an examination of the respiratory passageways with an instrument such as a rigid bronchoscope; certain types of surgery on the respiratory passageways; a gastrointestinal tract procedure, or a genitourinary tract procedure.

In a particular embodiment of the present invention, after a procedure that results in a lesion in the mouth of a subject, an effective amount of glycerophosphate salt is administered to the lesion to accelerate the healing of the lesion and prevent a disease or a condition related to an oral lesion. The effective amount of glycerophosphate salt can be administered in a form of dry powder, granule or gel to form a coating over the lesion, or in a mouthwash containing about 0.5% to about 75% by weight of a glycerophosphate salt. Procedures resulting in a mouth lesion include, but are not limited to, a routine daily activity such as chewing food, tooth brushing, or dental flossing; a dental procedure; an oral surgery such as tonsillectomy or adenoidectomy; an incision and drainage of infected oral tissue; an examination of the respiratory passageways with an instrument such as a rigid bronchoscope; and a surgery on the respiratory passageways or the gastrointestinal tract.

The dental procedures include, but are not limited to, a dental extraction; a periodontal procedure selected from the group consisting of surgery, scaling, root planting, probing, and recall maintenance; a dental implant placement; a reimplantation of an avulsed tooth; an endodontic instrumentation or surgery only beyond the apex; a subgingival placement of antibiotic fibers or strips; an initial placement of orthodontic bands but not brackets; an intraligamentary local anesthetic injection; a prophylactic cleaning of teeth or implant where bleeding is anticipated; a restorative dentistry with or without retraction cord; a local anesthetic injection; an intracanal endodontic treatment; a post placement and buildup; a placement of rubber dam; a postoperative suture removal; a placement of removable prosthodontic or orthodontic appliances; an orthodontic appliance adjustment; a taking of oral radiograph; and a shedding of primary teeth.

The application of a glycerophosphate salt to a lesion of the mouth to hasten the healing and sealing of same, thus to prevent a disease or a condition related to an oral lesion, can be concomitant with standard precautions normally taken under such circumstances. A glycerophosphate salt can be given in combination with one or more other drugs that can be used to treat or prevent a disease or a condition related to an oral lesion. The glycerophosphate salt and the other drugs can be administered simultaneously or sequentially, one following the other. When activities of the other drug, e.g., a certain antibiotic, are suppressed by a glycerophosphate salt, e.g., calcium glycerophosphate, the other drug is administered 1-2 hours apart from the administration of the glycerophosphate salt. The other drugs can be administered to the subject via routes of administration customarily used for such other drugs. The other drugs are not required to be administered to the lesion of the mouth together with the glycerophosphate salt.

In particular embodiments, a glycerophosphate salt can be administered in combination with one or more other agents. The other agents can be selected from the group consisting of, without limitation, (1) serotonin receptor antagonists; (2) serotonin receptor agonists; (3) histamine receptor antagonists; (4) bradykinin receptor antagonists; (5) kallikrein inhibitors; (6) tachykinin receptor antagonists, including neurokinin₁ and neurokinin₂ receptor subtype antagonists; (7) calcitonin gene-related peptide (CGRP) receptor antagonists; (8) interleukin receptor antagonists; (9) inhibitors of enzymes active in the synthetic pathway for arachidonic acid metabolites, including (a) phospholipase inhibitors, including PLA₂ isoform inhibitors and PLC_{γ} isoform inhibitors (b) cyclooxygenase inhibitors, and (c) lipooxygenase inhibitors; (10) prostanoid receptor antagonists including eicosanoid EP-1 and EP-4 receptor subtype antagonists and thromboxane receptor subtype antagonists; (11) leukotriene receptor antagonists including leukotriene B₄ receptor subtype antagonists and leukotriene D₄ receptor subtype antagonists; (12) opioid receptor agonists, including mu-opioid, delta-opioid, and kappa-opioid receptor subtype agonists; (13) purinoceptor agonists and antagonists including P_{2X} receptor antagonists and P_{2Y} receptor agonists; and (14) adenosine triphosphate (ATP)-sensitive potassium channel openers.

In another embodiment, a glycerophosphate salt can be administered in combination with one or more anti-infective agents. In a particular embodiment, a glycerophosphate salt can be administered in combination with a prophylactic antibiotic treatment. Prior to a procedure that involves manipulation of gingival tissue, the periapical region of teeth, or perforation of the oral mucosa, a subject who is considered to be a “high-risk patient” for endocarditis, can be subjected to a prophylactic antibiotic treatment. For example, about 30 to 60 minutes before the procedure, patients not allergic to penicillin can orally take about 2 grams (g) of cephalexin, cephradine or amoxicillin, or have about 1 g cefazolin or 2 g ampicillin via IM/IV; patients allergic to penicillin can take about 600 mg clindamycin orally or via IM/IV, or about 500 mg either azithromycin or clarithromycin orally; and an effective amount of glycerophosphate salt can be administered to the lesion after the procedure.

This invention will be better understood by reference to the non-limiting example that follows, but those skilled in the art will readily appreciate that the example is only illustrative of the invention.

### EXAMPLE

A tooth was removed from a human, male subject and normal packing was applied. There were five (5) stitches put in. Approximately 2 hours after the extraction, the human subject lightly rinsed his mouth and applied powdered CGP granulate of approximately 150 micron size granules directly to the gum and allowed the powder to become wet. Sufficient CGP powder was applied to form a light plaster coating over the wound. This was repeated approximately 6 hours later, before retiring that night, and again the next morning. In all cases, the CGP remained intact and in a cohesive mass on the gum for 15 minutes or more, after which time it slowly dissolved or dispersed and was safely swallowed. At no time during the post-extraction period was there swelling or pain. Self-observation by the human subject via mirror indicated that gums were normal pink color within 36-48 hours of the extraction. Rapid healing of the gum wounds was observed by a periodontist at the normal stitch-removal visit, 7 days subsequent to tooth removal. The gums had fully closed and were normal pink color in about one week. The periodontist commented that in his 15 years of practice, he had never seen this quality of healing at this speed.

### PRACTICAL APPLICATIONS OF THE PRESENT INVENTION

Methods according to embodiments of the present invention have been applied to multiple patients post implant placement. The age range of implant patients was 30-65 years. All patients had wound closure with 4-0 silk suture with interrupted knots unless otherwise noted. The patients were instructed to apply dry CGP that has less than 5% moisture in 150 micron granulate (PerioCell™, AkPharma Inc., Pleasantville, N.J. 08232) to the wounds three times daily, approximately 4 hours apart, for the first three days succeeding surgery. Mucosal soft tissue healing was observed and studied in these patients. Patient's compliance with the use instructions for CGP was reported by the patient at the post-operational visit (post-op) and was rated as Excellent, Good, Fair or Poor. In most, but not all cases, patients were reasonably compliant.

Patient 001 had 3 implants for mandibular overdenture and reported excellent compliance. At one week post-op, the periodontist noted excellent tissue approximation, very little erythema or edema, and no suppuration. At three week post-op, patient's closure was excellent, completely healed, and tissue was firm and pink.

Patient 002 had 4 implants for maxillary implant supported over denture and reported good compliance. At one week post-op, the periodontist noted a good tissue approximation, a slight gap at the right canine area, overall very good closure, and no suppuration. There was only mild erythema at the wound margin. At the four week post-op, the periodontist was pleased to note a complete heal with total wound approximation.

Patient 003 had 5 implants for a fixed mandibular hybrid prosthesis and reported good compliance. At one week post-op, the periodontist noted excellent wound closure with great tissue approximation and no suppuration. The most distal implant on the left side had a slight cover screw exposure. Everything else was submerged. There was mild erythema at the wound margins. At four week post op, the periodontist was pleased to note that the wound was completely healed with pink healthy tissue noted. All implants were then submerged.

Patient 004 had 1 implant in the upper left premolar area and reported good compliance. At one week post-op, the periodontist was pleased with a beautiful wound approximation. There was only mild erythema with mild edema at the margins. No suppuration was noted. At the one month post-op, the periodontist noted a complete heal with no erythema or edema. Complete implant coverage was achieved.

Patient 005 had 1 implant in the lower left first molar area and reported excellent compliance. At one week post-op, the periodontist noted perfect wound margin approximation with very little gingival erythema noted. The implant was completely covered. No suppuration was noted. At one month post-op, patient's healing was complete. The margins were tight with totally submerged implant noted.

Patient 006 had 2 implants in the lower left pre-molar area and 1 implant in the lower right molar area and reported fair compliance. At one week post-op, the periodontist noted moderate tissue gaps in the left side. Both implant cover screws were exposed. Slight bony exposure was observed. The right side was a bit better with only slight tissue gapping noted. Both sides had moderate erythema at the wound margin. At 1 month post-op, things looked better with still slight cover screw exposure on the left side. The margins were slightly rolled.

Patient 007 had 2 implants in the lower right premolar area and reported good compliance. At one week post-op, the periodontist was pleased that the wound margins came together very well with no gaps noted. There was mild erythema at the margins with no suppuration noted. The one month post op showed a perfectly closed wound with pink and healthy gingival issues noted. Implants were both submerged.

Patient 008 had one implant in the upper right premolar area and reported fair compliance. At one week post op, the periodontist noted greater than 50% gapping of the margins. This patient admitted to smoking 2 cigars per day which delays soft and hard tissue wound healing. There was moderate erythema at the wound margins with a pin point area of suppuration noted. After 3 weeks this patient's implant was removed and deemed a smoking failure. A bone graft was placed at the site with strict orders of NO Smoking.

Patient 009 had one implant in the lower right premolar area and reported good compliance. At one week post-op, the periodontist noted great wound approximation with no gaps. Mild erythema noted at the wound margin. No suppuration and complete coverage of the implant was achieved. The one month post-op revealed perfect tissue approximation. Pink healthy gingival tissue was noted.

Patient 010 had 2 implants in the upper left posterior area and reported excellent compliance. At the one week post op, the periodontist was very pleased with great wound approximation. No gaps were noted and the implants were both completely submerged. Only localized mild erythema was noted at the wound margins. At one month post-op, the periodontist was pleased with the patient's perfect wound approximation. No gaps were noted. Pink healthy tissue was observed.

Patient 011 had 1 implant in the lower left molar area and reported fair compliance. Overall at one week post-op, patient's healing went well. There was a slight tissue gap at the mesial aspect leading to a slight cover screw exposure. The rest of the wound was closed well. Mild erythema of the tissue margins was noted. One month post-op looked great. There were no gaps, no erythema or edema at the margins, and no discomfort.

Patient 012 had 2 implants in the lower right molar area and reported good compliance. At the one week post-op there were no gaps seen with little to no marginal erythema. No suppuration was noted. One month post-op revealed excellent healing with no erythema or edema noted. There was a slight exposure of the distal implant's cover screw. Mesial implant was completely covered. No discomfort was reported.

Patient 013 had 3 implants in the lower anterior for an implant supported over denture. Patient admitted to non-compliance. At one week post-op, the periodontist noted very poor tissue presentation. There was complete gapping of the entire wound with significant bony exposure. The wound margins were moderately erythematous. Poor tissue heal was observed. One week later, things looked a bit better with some granulation over the bone but the healing was still slow. Significant bone was still exposed. Things were improving slowly but bony exposure was still present especially on the left side. About three weeks post-op, things were finally much improved. No bony exposure with cover screw exposure present on the middle and right implant. Patient had significant pain throughout this healing process.

Patient 014 had 3 implants in the maxillary anterior and reported excellent compliance. At one week post-op, the periodontist was so pleased with excellent wound approximation and perfect closure. The patient was moderately bruised extraorally, however, the wound margins showed almost no erythema. Implants were all covered. One month check showed perfect closure with all implants submerged. Slight erythema was noted at the distal aspect of the right die of the wound edge, probably due to temporary impingement. No discomfort was reported.

Patient 015 had 2 implants in the lower left posterior and reported good compliance. At one week post-op, the periodontist was pleased with a great heal. No gaps were noted. Mild erythema with no suppuration was observed. Both implants were submerged. One month check revealed that about half of the mesial implant had a cover screw exposed but the distal implant was completely submerged. Overall good approximation and no erythema were observed. No discomfort was reported.

Patient 016 had 2 implants in the lower left molar area and reported excellent compliance. One week post-op revealed excellent wound approximation with all implants submerged. There was only mild erythema at the margins at the distal aspect of the wound. No suppuration was noted.

Patient 017 had 2 implants placed with one in the upper right and the other in the lower left, and reported good compliance. Wound closure was better in the mandible than in the maxilla. There were some edge gapping in the maxilla, slight cover screw exposure, and moderate marginal erythema. The mandibular wound was closed better with no gap noted. Erythema was less and the implant was totally covered. Only mild discomfort was noted during the week. At one month post op, all looked great. Mandibular wound was closed perfectly. There was no erythema or edema. There was slight cover screw exposed on the maxillary implant, but the wound was totally healed with no erythema noted.

Patient 018 had 1 implant in the lower right posterior and reported good compliance. Wound closure looked great with no gaps noted at a 2 week post op. Implant was completely covered. This wound was closed with a chromic gut suture due to the fact that the periodontist was going away on vacation the next week. There was mild mesial inflammation at the margins at 2 weeks with no suppuration noted. Patient described only mild discomfort over the first week of healing and no discomfort at 2 weeks. The one month post-op looked great with complete closure noted. No gaps and no erythema or edema were noted.

Patient 019 had 1 implant in the maxillary canine area and reported poor compliance. One week post op revealed moderate gap at wound margin with rolled borders. Granulation tissue was filling the gaps therefore no part of the cover screw was exposed. Moderate erythema was noted. Mild discomfort was reported at one week. One month post op revealed good closure. No gaps were noted with the implant completely submerged. Mild erythema was noted at the mesial aspect due to plaque accumulation on the lateral incisor. No discomfort was reported.

Patient 020 had 1 implant in the upper right posterior and reported good to fair compliance. One week post-op revealed a mild gap at the wound margin with slight cover screw exposure noted. Patient is a smoker, which makes breakdown of a maxillary wound more commonly to occur. No suppuration was noted. Only mild discomfort was noted. Moderate erythema was noted at the wound margin. One month check revealed slight cover screw exposure. Overall wound margin looked good. Still some mild erythema was noted but no discomfort was reported.

Patient 021 had 2 implants in the maxillary right posterior and reported good compliance. Patient had poor oral hygiene. One week post op revealed good wound approximation with implants completely submerged. Borders were slightly rolled. 2 clots were present at wound margins as patient was on high doses of Coumadin. Moderate erythema was noted at the mesial and distal aspects of the wound margin—possibly due to the fact that there was moderate plaque accumulation on the adjacent teeth. One month check looked better as patient's hygiene had improved slightly. All implants were covered and the wound margins were completely approximated. No discomfort was noted.

Patient 022 had 2 implants in the mandibular right posterior and reported excellent compliance. One week post op revealed excellent wound approximation with all implants completely submerged. Mild to no discomfort was noted. There was mild bruising to the chin. Mild marginal erythema was noted. Patient had good oral hygiene. No suppuration was noted. 3 week post op revealed excellent heal with full coverage of implants. No marginal erythema or discomfort was noted.

Patient 023 had 2 implants in the maxillary left posterior with 1 implant in the maxillary right posterior, and reported good compliance. One week post-op revealed better healing on the left side than on the right. No gaps were observed on the left with full implant coverage noted. The right side site was between 2 natural teeth and the left side was a distal extension. The right side site showed mild gapping but no cover screw exposure as granulation tissue was filling the gap. Mild left side erythema and more moderate right side marginal erythema were noted. No discomfort was noted. 3 week post op showed a perfect heal on the left side with great wound approximation. There was a slight cover screw exposure on the right side. Mild right side erythema was noted.

Patient 024 had 2 implants maxillary left posterior and reported good compliance. One week post-op revealed a slight gap at the wound margin. Margin was rolled but no implant cover screws exposed as granulation tissue was filling in the gap. Mild to moderate marginal erythema was noted. Patient had good to fair oral hygiene. Mild discomfort was noted for first few days followed by no discomfort at one week. One month post op has yet to be performed.

Patient 025 had 1 implant in the maxillary right posterior and reported good compliance. One week post-op revealed excellent wound approximation. Mild erythema was noted. Patient had good oral hygiene. Mild, but improving, discomfort was noted. Implants were totally submerged. No suppuration was noted. One month post op has yet to be performed.

Patient 026 had 1 implant in the maxillary left posterior and reported fair compliance. Patient is a smoker. One week post-op revealed rolled wound borders with slight gapping across. Slight cover screw was exposure. Mild marginal erythema noted. No suppuration was noted. There was mild one week discomfort. Patient had good to fair oral hygiene. One month post op has yet to be performed.

With regard to implant healing and overall implant success, there was a direct correlation with wound healing. The goal in a two stage procedure is to achieve a tight wound approximation with as few gaps as possible. Patients with a good heal with few to no tissue gaps generally reported little to no post-op discomfort. This usually correlated with a high implant success rate. Patients with more moderate to severe gapping often had more post-op pain and swelling post-operatively.

The periodontist who conducted the above small and uncontrolled study believed that the results from the study had important significance. A definite correlation was observed between good and excellent compliance with the use instructions for CGP and the little to no tissue gapping and in turn good wound healing. For example, in general, patients with excellent compliance had no or very little erythema, less gaps, and good wound closure at much earlier time. Patients with less compliance most definitely showed a trend towards tissue gaps and slower wound healing. Overall, mandibular wounds appeared to heal better than maxillary wounds. This is possibly related to the fact that CGP sits better on the mandible (gravity) or because the tissue in the maxilla is more bound down and therefore harder to close.

As is frequently the case in any study that requires patient compliance, the determination as to whether it is the tested agent that is primarily responsible for the results or whether it is faithful use of the agent that is in and of itself a marker for patient overall good self-care habits is contributory to outcome. It is apparent that certain factors can be confounding with regard to tissue healing post implant placement. Smoking is a major player which most definitely adds to poor wound healing as well as an increased risk of implant non-integration. Diabetic status is also a big player. Diabetics with poor sugar control have inherent chemotactic defects and thus poor wound healing. In addition, oral hygiene also comes into play.

The periodontist noted two most significant results from the study of the post-op periodontal implant patients who used PerioCell™ (CGP). First, quicker wound closure and wound approximation were observed. The use of PerioCell shortened healing time by about 1.5 to 2 weeks (30%-50% improvement). Second, decreased marginal wound erythema was observed. There was a direct correlation between tissue coverage and implant success rate.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method of improving healing of an oral lesion in a subject, the method comprising:
(a) locating the oral lesion in the mouth of the subject; and
(b) applying an effective amount of a glycerophosphate salt to the oral lesion in a form of dry powder, granule or gel to form a coating over the oral lesion, or in a mouthwash containing about 0.5% to about 75% by weight of a glycerophosphate salt.

2. The method of claim 1, wherein the glycerophosphate salt is administered to the oral lesion in a form of dry powder, granule or gel to form a coating over the oral lesion, and wherein the coating contains about 25% to 100% by weight of the glycerophosphate salt.

3. The method of claim 1, wherein the oral lesion is located on a gum, inner cheek, throat, palate, tongue, or lips.

4. The method of claim 1, wherein the glycerophosphate salt is selected from the group consisting of calcium glycerophosphate, magnesium glycerophosphate, zinc glycerophosphate, manganese glycerophosphate, lithium glycerophosphate, cupric glycerophosphate, ferric glycerophosphate, quinine glycerophosphate, glycerophosphate disodium; glycerophosphate dipotassium, glycerophosphate barium, glycerophosphate strontium, and combinations thereof.

5. The method of claim 4, wherein the glycerophosphate salt is calcium glycerophosphate.

6. The method of claim 1, wherein the subject is a human subject.

7. A method of preventing a disease or condition related to an oral lesion in a subject, the method comprising improving healing of the oral lesion in the subject according to a method of claim 1.

8. The method of claim 7, wherein the disease or condition related to an oral lesion is a mouth disease or condition, a cardiovascular disease or condition, a poorly controlled diabetes, or preterm birth.

9. The method of claim 8, wherein the mouth disease or condition is gingivitis, periodontitis, acute necrotizing ulcerative gingivitis, or a sore.

10. The method of claim 8, wherein the cardiovascular disease or condition is endocarditis, stroke, atherosclerotic plaques in the arteries, or mitral valve prolapse.

11. The method of claim 7, wherein the disease or condition related to an oral lesion is oral mucositis.

12. The method of claim 7, wherein the subject has a higher risk of a disease or condition related to an oral lesion.

13. The method of claim 12, wherein the subject suffers from a preexisting heart condition, diabetes or another endocrine disorder, an immune disorder, a sexually transmitted disease, a virus infection, an inflammatory bowel disease, neutropenia, a blood disorder, a trauma, an accident of all types, a war wound, a barroom brawl jaw punch, or a recurrent oral skin problem.

14. The method of claim 13, wherein the preexisting heart condition is selected from the group consisting of an artificial heart valve, a history of previous endocarditis, a damaged heart valve, a congenital heart, a heart valve defect, hypertrophic cardiomyopathy, a surgically constructed systemic-pulmonary shunt, and a mitral valve prolapse.

15. The method of claim 12, wherein the subject is under radiation, chemotherapy or a treatment of an immune-suppression drug.

16. The method of claim 15, wherein the subject suffers from oral mucositis.

17. The method of claim 12, wherein the subject is undergoing a procedure that predisposes the subject to a disease or condition related to an oral lesion, the procedure is selected from the group consisting of an implant procedure; a total joint replacement procedure; a dialysis therapy; a dental procedure; an oral surgery; an incision and drainage of infected oral tissue; an examination of the respiratory passageways with a rigid instrument; a surgery on the respiratory passageways; a gastrointestinal tract procedure; and a genitourinary tract procedure.

18. The method of claim 17, wherein before the procedure, the subject receives a prophylactic treatment with an antibiotic selected from the group consisting of cephalexin, cephradine, amoxicillin, cefazolin, ampicillin, clindamycin, azithromycin and clarithromycin.

19. The method of claim 1, wherein the subject receives one or more additional agents selected from the group consisting of an analgesic, an anti-inflammatory agent, an anti-infective agent, an anti-diabetic agent, and combinations thereof.

20. The method of claim 1, wherein the oral lesion results from a procedure selected from the group consisting of a routine daily activity; a dental procedure; an oral surgery; an incision and drainage of infected oral tissue; an examination of the respiratory passageways with a rigid instrument; and a surgery on the respiratory passageways.

21. The method of claim 20, wherein the routine daily activity is chewing food, tooth brushing, or dental flossing.

22. The method of claim 20, wherein the dental procedure is selected from the group consisting of a dental extraction; a periodontal procedure selected from the group consisting of surgery, scaling, root planting, probing, and recall maintenance; a dental implant placement; a reimplantation of avulsed tooth; an endodontic instrumentation or surgery only beyond the apex; a subgingival placement of antibiotic fibers or strips; an initial placement of orthodontic bands but not brackets; an intraligamentary local anesthetic injection; a prophylactic cleaning of teeth or implant when bleeding is anticipated; a restorative dentistry with or without retraction cord; a local anesthetic injection; an intracanal endodontic treatment; a post placement and buildup; a placement of rubber dam; a postoperative suture removal; a placement of removable prosthodontic or orthodontic appliances; an orthodontic appliance adjustment; a taking of oral radiograph; and a shedding of primary teeth.

23. The method of claim 20, wherein the oral surgery is tonsillectomy or adenoidectomy.

24. The method of claim 1, wherein the oral lesion is associated with oral mucositis.

25. A kit for improving healing of an oral lesion in a subject, the kit comprising a glycerophosphate salt and instructions for using the glycerophosphate salt to improve healing of an oral lesion in a subject.
